(19) United States
(12) Patent Application Publication
(10) Pub. No.: US 2002/0009507 A1
(43) Pub. Date: Jan. 24, 2002

(54) Use of polyethoxylated castor oil for the treatment of dry eye

(75) Inventors: Lori K. Weimer, The Woodlands, TX (US); Daniel A. Gamache, Arlington, TX (US)

Correspondence Address: ALCON RESEARCH, LTD., R&D COUNSEL, Q-148, 6201 SOUTH FREEWAY, FORT WORTH, TX, 76134-2099, US

(73) Assignee: Alcon Universal Ltd.

(21) Appl. No.: 09/757,184

(22) Filed: Jan. 9, 2001

Related U.S. Application Data
(60) Provisional application No. 60/177,060, filed on Jan. 19, 2000.

(51) Int. Cl. A61K 35/78

(52) U.S. Cl. 424/731

(57) ABSTRACT

The use of polyethoxylated castor oils to treat dry eye disorders is disclosed.

This application claims priority to co-pending U.S. Provisional Application, U.S. Ser. No. 60/177,060, filed Jan. 19, 2000.

### BACKGROUND OF THE INVENTION

The present invention relates generally to a method for treating dry eye disorders. In particular, the present invention relates to the use of certain polyethoxylated castor oil compounds to treat dry eye.

Dry eye, also known generically as keratoconjunctivitis sicca, is a common ophthalmic disorder affecting millions of Americans each year. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. In some cases, dry eye complications are associated with other diseases, such as Sjogren's disease and cicatricial pemphigoid.

Although it appears that dry eye may result from a number of unrelated pathogenic causes, all presentations of the complication share a common effect, that is the breakdown of the pre-ocular tear film, which results in dehydration of the exposed outer surface and many of the symptoms outlined above (Lemp, Report of the Nation Eye Institute/Industry Workshop on Clinical Trials in Dry Eyes, The CLAO Journal, volume 21, number 4, pages 221-231 (1995)).

Practitioners have taken several approaches to the treatment of dry eye. One common approach has been to supplement and stabilize the ocular tear film using so-called artificial tears instilled throughout the day. Another approach has been the use of ocular inserts that provide a tear substitute or to stimulate endogenous tear production. Still another approach involves plugging the lacrimal ducts so that tears are retained in the conjunctival cul-de-sac.

Examples of the tear substitution approach include the use of buffered, isotonic, viscous saline solutions. Tear reconstitution is also attempted by providing one or more components of the tear film such as phospholipids and oils. Examples of these treatment approaches are disclosed in U.S. Pat. No. 4,131,651 (Shah et al.); U.S. Pat. No. 4,370,325 (Packman); U.S. Pat. No. 4,409,205 (Shively); U.S. Pat. Nos. 4,744,980 and 4,883,658 (Holly); U.S. Pat. No. 4,914,088 (Glonek); U.S. Pat. No. 5,075,104 (Gressel et al.); U.S. Pat. No. 5,294,607 (Glonek et al.); and U.S. Pat. No. 5,403,841 (Lang). U.S. Pat. No. 4,818,537 (Guo) discloses the use of a lubricating, liposome-based composition.

Aside from the above efforts, which are directed primarily to the alleviation of symptoms associated with dry eye, methods and compositions directed to treatment of the dry eye condition have also been pursued. For example, U.S. Pat. No. 5,041,434 (Lubkin) discloses the use of sex steroids, such as conjugated estrogens, to treat dry eye condition in post-menopausal women; U.S. Pat. No. 5,290,572 (MacKeen) discloses the use of finely divided calcium ion compositions to stimulate preocular tear film; U.S. Pat. No. 4,966,773 (Gressel et al.) discloses the use of microfine particles of one or more retinoids for ocular tissue normalization; and U.S. Pat. No. 5,696,166 (Yanni et al.) discloses the use of hydroxyeicosatetraenoic acid derivatives for stimulating mucin production in the eye.

The use of non-ionic surfactants, such as polyethoxylated castor oil compounds, as solubilizing agents for pharmaceutically active compounds is known. See, for example, U.S. Pat. No. 4,960,799 (Nagy) in which a polyethoxylated castor oil is used as a solubilizing agent in compositions containing the non-steroidal drug diclofenac. The use of non-ionic surfactants such as polyethoxylated castor oils in stable emulsions is also known. U.S. Pat. No. 4,075,333 (Josse) discloses stable, intravenous emulsion formulations of vitamins. El-Sayed et al., Int J. Pharm., 13:303-12 (1983) discloses stable oil-in-water emulsions of an antineoplastic drug. U.S. Pat. No. 5,185,372 (Ushio et al.) discloses topically administrable ophthalmic formulations of vitamin A in which a non-ionic surfactant is used to form an emulsion of vitamin A in an aqueous medium.

U.S. Pat. Nos. 5,631,287 and 5,849,792 (Schneider) disclose aqueous prostaglandin compositions containing polyethoxylated castor oil compounds, including Cremophor EL, as stabilizing agents for prostaglandin drugs.

U.S. Pat. No. 5,686,488 (Gamache, et al.) discloses the use of polyethoxylated castor oil products for treating inflammatory disorders. Topical ocular solutions containing a polyethoxylated castor oil compound as an active ingredient are disclosed. The '488 patent does not mention dry eye disorders.

### SUMMARY OF THE INVENTION

The present invention is directed to the use of polyethoxylated castor oil compounds in pharmaceutical compositions intended for the treatment of dry eye disorders. Dry eye is not necessarily considered an inflammatory condition of the eye. Dry eye can be caused by a variety of etiologies, including environmental (e.g., low-humidity, video display terminal use) and anatomical (e.g., blink disorders) factors. Regardless of cause, dry eye is characterized by abnormal tear composition resulting in discomfort and ocular surface damage. Corneal injury that occurs in dry eye may be distinguished from inflammation because unlike inflammation it is not necessarily associated with leukocytes, flare or swelling. Without wishing to be bound by any theory, it is believed that polyethoxylated castor oil compounds mimic or enhance tear composition.

### DETAILED DESCRIPTION OF THE INVENTION

The polyethoxylated castor oil compounds useful in the methods of the present invention are commercially available, and include those classified as PEG-2 to PEG-200 castor oils, as well as those classified as PEG-5 to PEG-200 hydrogenated castor oils. Such polyethoxylated castor oils include those manufactured by Rhone-Poulenc (Cranbury, N.J.) under the Alkamuls® brand, those manufactured by BASF (Parsippany, N.J.) under the Cremophor® brand, and those manufactured by Nikko Chemical Co., Ltd. (Tokyo, Japan) under the Nikkol brand. Preferred polyethoxylated castor oils are those classified as PEG-15 to PEG-50 castor oils, and more preferred are PEG-30 to PEG-35 castor oils. It is most preferred to use those polyethoxylated castor oils known as Cremophor® EL and Alkamuls® EL-620. Preferred polyethoxylated hydrogenated castor oils are those classified as PEG-25 to PEG-55 hydrogenated castor oils. The most preferred polyethoxylated hydrogenated castor oil is PEG-40 hydrogenated castor oil, such as Nikkol HCO-40.

In general, the methods of the present invention comprise the topical administration of an aqueous composition comprising one or more polyethoxylated castor oil compounds. The compositions used in the methods of the present invention will include one or more polyethoxylated castor oils in an amount from about 0.02-20 % by weight (wt %). It is preferred to use one or more polyethoxylated castor oils in an amount from about 0.1-5 wt %, and it is especially preferred to use an amount from about 0.5-2 wt %.

In addition to the polyethoxylated castor oil compound(s), the compositions of the present invention may further comprise various excipients, such as antimicrobial preservatives, tonicity agents, and buffers. Examples of suitable antimicrobial preservatives include: benzalkonium chloride, thimerosal, chlorobutanol, methyl paraben, propyl paraben, phenylethyl alcohol, edetate disodium, sorbic acid, polyquaternium-1 and other agents known to those skilled in the art. Such preservatives, if utilized, will typically be employed in an amount from about 0.001-1 wt %. Examples of suitable agents for adjusting the tonicity or osmolality of the formulations include sodium chloride, potassium chloride, mannitol, dextrose, glycerine and propylene glycol. Such agents, if utilized, will be employed in an amount from about 0.1-10 wt %. Examples of suitable buffering agents include acetic acid, citric acid, carbonic acid, phosphoric acid, boric acid, the pharmaceutically acceptable salts of the foregoing, and tromethamine. Such buffers, if utilized, will be employed in an amount from about 0.001-1 wt %.

The compositions are preferably aqueous, have a pH from about 5 to 8, and an osmolality between about 260 to 320 milliOsmoles per kilogram (mOsm/kg).

### EXAMPLE 1

The following topically administrable ophthalmic formulations are representative of the compositions used in the methods of the present invention. All amounts in Table 1 are expressed in units of % (w/v).

TABLE 1

| INGREDIENT | A | B |
|---|---|---|
| Polyethoxylated Castor Oil | 0.1-5 | 0.1-5 |
| Sodium Chloride | 0.52 | 0.6 |
| Potassium Chloride | 0.12 | 0.12 |
| Hydroxypropyl Methylcellulose | 0.3 | 0.3 |
| (2910) | | |
| Sodium Borate (decahydrate) | 0.35 | 0.5 |
| Polyquaternium-1 | — | 0.0011 |
| HCl/NaOH | q.s. pH 7.5 | q.s. pH 7.4 |
| Purified Water | q.s. 100 | q.s. 100 |

### EXAMPLE 2

The dry eye activity of four formulations was evaluated in a rabbit desiccation model (Nakamura et al., Experimental Eye Research 65(4), 569 -574 (1997)). According to this model, the cornea of an anesthetized rabbit is desiccated for 4 hours, using a speculum to keep the eye open. The dried cornea is then stained with methylene blue to determine the desiccation-induced corneal epithelial damage. The cornea is removed and an 8 mm circular punch of the central cornea is collected. The corneal punch is placed into a solution of acetone/ammonium sulfate to extract the methylene blue dye from the tissue. Absorbance readings are then taken on a spectrophotometer (660 nm wavelength).

For the naive controls, rabbits are anesthetized and the methylene blue is immediately applied. To test a formulation's protective effects, 50 ul of the test article is applied topically and the eyelids are taped shut for 10 minutes. After this time, the tape is removed and the speculum inserted. The cornea is then desiccated for 4 hours, as described previously. If the tested formulation protects the cornea from desiccation, the uptake of methylene blue dye into the cornea is reduced compared to that of the vehicle control. Percent protection is determined by the formula:

(1−(experimental absorbance/naive absorbance)/(vehicle control absorbance/naive absorbance))*100.

The results are shown in Tables 2 and 3 below.

TABLE 2

| | Methylene Blue | |
|---|---|---|
| Test Group | Absorbance | % Protection |
| Naïve | 0.273 ± 0.081 | — |
| Vehicle Control^{a} | 0.674 ± 0.223 | — |
| VOLTAREN OPHTHAMIC ® | 0.318 ± 0.069 | 89^{b} |
| Diclofenac Sodium Sterile | | |
| Solution 0.1% (CIBA Vision | | |
| Corp.) | | |
| Diclofenac Sodium Ophthalmic | 0.572 ± 0.091 | 25 |
| Solution 0.1% | | |
| (FALCON ® Ophthalmics, Inc.) | | |

^{a}Vehicle Control = BSS ® (Balanced Salt Solution) Sterile Irrigating Solution [Alcon Laboratories, Inc.] +0.064% (v/v) ethanol added.
^{b}Statistically significant difference compared to vehicle control as determined by Student's t-test (p < 0.05).

TABLE 3

| Test Group | Methylene Blue Absorbance | % Protection |
|---|---|---|
| Naïve | 0.271 ± 0.081 | — |
| Vehicle Control^{a} | 0.726 ± 0.259 | — |
| Cremophor EL ®^{c} | 0.447 ± 0.106 | 61^{b} |

^{a,b}same as above.
^{c}Vehicle Control + 5.0% (v/v) Cremophor EL ® polyethoxylated castor oil

The results shown in Tables 2 and 3 demonstrate that formulations containing polyethoxylated castor oils are effective in treating dry eye. The superior performance of VOLTAREN Ophthalmic® Diclofenac Sodium Sterile Solution 0.1% compared to Diclofenac Sodium Ophthalmic Solution 0.1% shown in Table 2 is attributable to the polyethoxylated castor oil ingredient in the VOLTAREN Ophthalmic® product. Both diclofenac products contain the same concentration of active (0.1% diclofenac sodium), but VOLTAREN Ophthalmice contains a polyethoxylated castor oil ingredient (polyoxyl 35 castor oil) whereas the Diclofenac Sodium Ophthalmic Solution 0.1% product does not contain any polyethoxylated castor oil ingredient. This result is confirmed in Table 3 where a polyethoxylated castor oil ingredient alone is added to Vehicle Control.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A method of treating dry eye disorders comprising the topical ophthalmic administration of an aqueous pharmaceutical composition comprising a polyethoxylated castor oil as the sole active ingredient.

2. The method of claim 1 wherein the polyethoxylated castor oil is present at a concentration from about 0.02-20 wt %.

3. The method of claim 2 wherein the polyethoxylated castor oil is present at a concentration from about 0.1-5 wt %.

4. The method of claim 3 wherein the polyethoxylated castor oil is present at a concentration from about 0.5-2 wt %.

5. The method of claim 1 wherein the polyethoxylated castor oil is selected from the group consisting of: PEG-2 to PEG-200 castor oils and PEG-5 to PEG-200 hydrogenated castor oils.

6. The method of claim 5 wherein the polyethoxylated castor oil is selected from the group consisting of: PEG-1 5 to PEG-50 castor oils and PEG-25 to PEG-55 hydrogenated castor oils.

7. The method of claim 6 wherein the polyethoxylated castor oil is selected from the group consisting of: PEG-30 to PEG-35 castor oils and PEG-40 hydrogenated castor oil.

8. The method of claim 1 wherein the composition further comprises a preservative, a tonicity adjusting agent and a buffer.

9. The method of claim 8 wherein the composition has a pH from about 5 to 8 and an osmolality between about 260 to 320 mOsm/kg.
